(19) United States
(12) Patent Application Publication
(10) Pub. No.: US 2026/0144727 A1
(43) Pub. Date: May 28, 2026

(54) FATTY ACID DERIVATIVE, PRODUCTION PROCESS THEREFOR, HAIR COMPOSITION AND HAIR-TREATMENT METHOD

(71) Applicant: CHEMYUNION LTDA., Sorocaba (BR)

(72) Inventors: Cleverson Rogério Princival, Sorocaba (BR); Marcos Roberto Rossan, Sorocaba (BR); Lilian Mussi, Sorocaba (BR); Flávio Bueno De Camargo Junior, Sorocaba (BR); Wagner Vidal Magalhães, Sorocaba (BR); Andrew Thomaz Camillo, Sorocaba (BR); Edson Katekawa, Sorocaba (BR)

(73) Assignee: CHEMYUNION LTDA., Sorocaba (BR)

(21) Appl. No.: 19/453,714

(22) Filed: Jan. 20, 2026

Related U.S. Application Data
(62) Division of application No. 18/004,596, now abandoned.

(30) Foreign Application Priority Data
Jul. 7, 2020 (BR) 1020200138553

(51) Int. Cl. A61K 8/42; A61Q 5/12; C07C 231/02; C07C 233/20; C12P 13/02

(52) U.S. Cl. CPC A61K 8/42; A61Q 5/12; C07C 231/02; C07C 233/20; C12P 13/02

(57) ABSTRACT

A hair composition, a method of hair treatment, a process of producing fatty acid derivatives, and the use of these hair compositions, the composition having fatty hydroxy esters to improve hair shine and strength.

### CROSS REFERENCE TO RELATED APPLICATIONS

This application is a divisional of U.S. application Ser. No. 18/004,596, filed Jan. 6, 2023, which is a U.S. National Phase of International Application No. PCT/BR2021/050294, filed Jul. 7, 2021, which claims the benefit of Brazilian Provisional Application No. 10 2020 013855 3, filed Jul. 7, 2020.

### BACKGROUND OF THE INVENTION

### Technical Field

The present invention describes a hair composition, a method of hair treatment, a process of producing fatty acid derivatives, and the use of these hair compositions. More specifically, the present invention comprises fatty hydroxy esters in hair compositions to improve hair shine and strength. The present invention is located in the fields of Personal Hygiene, Cosmetics and Chemistry.

### Prior Art

Hair is constantly damaged through washing, dyeing, bleaching and heat treatments. These procedures, most of the time, lead to changes in the structures of the hair fiber, causing damage, mainly to the cuticle (region present on the surface of the hair fiber) responsible for the characteristics of shine and smoothness of the hair.

Silicone-based compounds have been used in cosmetic formulations for hair to promote an increase in hair fiber shine. The most used silicones in the cosmetic area are phenyltrimethicone, cyclomethicone, dimethicone and amino propyl phenyltrimethicone. However, silicones are obtained by chemical reactions involving chlorinated compounds of petrochemical origin and non-renewable source, that offer environmental danger, as well as are related to their bioaccumulative properties, making the threads heavy and difficult to model and have low biodegradability. Recently, the cosmetic market has been looking for new alternatives to produce more sustainable, biodegradable and environmentally attractive products.

In the search for the state of the art, in scientific and patent literatures, the following documents were found that deal with the subject:
(1) Atkins, P. W. Physical Chemistry 8th ed, Oxford. 2006. (2) Bhushan, B. Principles of Tribology, CRC Press. 2000.
(3) Nelson, D. L.; Cox, M. M. Principles of Biochemistry 7 ed. ARTMED. 2018.
(4) REICH, C.; ROBBINS, C. R., Light scattering and shine measurements of human hair: A sensitive probe of the hair surface. Journal of Cosmetics Science, v. 44, 1993, 221-234.
(5) Product Information Personal Care Dow Corning® 556 Cosmetic Grade Fluid. 2014.
(6) Anthony J. O'Lenick Jr. Silicones for Personal Care. Allured Publishing Corporation. 2 ed. 2008.
(7) Lassen, C.; Hansen, C. L.; Mikkelsen, S. H.; Maag, J. Siloxanes-Consumption Toxicity and Alternatives. COWI A/S. 2005.
(8) Schaefer, K. Silicones in Hair Care: Making Innovative Solutions Possible. Cosmetics & Toiletries. 2020, 1-4.
(9) Goddard, E. D.; Gruber, J. V. Principles of Polymer Science and Technology in Cosmetics and Personal Care. CRC Press, 1999.
(10) Bom. S.; Jorge, J.; H, Ribeiro, M.; Marto, J. A step forward on sustainability in the cosmetics industry: A review. Journal of Cleaner Production. 2019, 271-284.
(11) DOC Japan co. ltd. Cosmetic conditioning oil composition and cosmetic product U.S. Pat. No. 9,987,210 B2, 5 Jun. 2018.
(12) Hans-Heinrich, M.; Manfred, S. M.; Gebhard, W. Silicones, Ullmann's Encyclopedia of Industrial Chemistry. Wiley-VCH, 2012
(13) Nagase, S. Hair Structures Affecting Hair Appearance, Cosmetics, 6, 2019, 1-15
(14) CRODA INTERNATIONAL PLC. Hair care formulation. WO 2016/189276 AI, 1 Dec. 2016.
(15) Carey, F. A.; Sundberg, R. J. Advanced Organic Chemistry: Part A: Structure and Mechanisms 5 ed. 2008.
(16) Carey, F. A.; Sundberg, R. J. Advanced Organic Chemistry: Part B: Structure and Mechanisms 5 ed. 2008.
(17) Climent, M. J.; Corma, A.; Iborra, S. Heterogeneous Catalysts for the One-Pot Synthesis of Chemicals and Fine Chemicals. Chem. Rev. 2011, 111, 1072-1133
(18) Talukder, R. M. M.; Wu, J. C.; Lau, S. K.; Cui, L. C. Shimin, G.; Lim, A. Comparison of Novozym 435 and Amberlyst 15 as Heterogeneous Catalyst for Production of Biodiesel from Palm Fatty Acid Distillate. Energy & Fuels, Vol. 23, 2009, 1-4.
(19) Zhang, K.; Li, W.; Chen, H.; Wang, Y.; Yang, B. Lipase-catalyzed synthesis of 1,3-propanediol monoester in a solvent-free system. Zhongguo Youzhi. 2010, 35, 28-31.
(20) Whittall, J.; Sutton, P. W.; Kroutil, Wolfgang. Practical Methods for Biocatalysis and Biotransformations. Wiley. 2016.
(21) Hsiao-Ching C.; Chia-Hung, K. Wei-Chuan, T.; Yi-Lin, C.; Wen-Dee, C.; Chieh-Ming J.; Yung-Chuan, L.; Chwen-Jen, S. Product Selectivity and Optimization of Lipase-Catalyzed 1,3-Propylene Glycol Esters by Mixture Design and RSM J Am Oil Chem Soc. 2012, 89, 231-241.
(22) PCPC on-line INFOBASE. Available in: https://online.personalcarecouncil.org/jsp/IngredientDetail.jsp?monoid=2713. Access at Sep. 3, 2020
(23) LEFAUDEUX, N.; LECHOCINSKI, N.; CLEMENCEAU, P.; BREUGNOT, S. New luster formula for the characterization of hair tresses using polarization imaging. Third Annual Conference on Applied Hair Science, 2008.
(24) GAO, T.; PEREIRA, A.; ZHU, S. Study of hair shine and hair surface smoothness. Journal of Cosmetics Science, v. 60, 2009, 187-197.

Thus, from what can be seen from the researched literature, no documents were found anticipating or suggesting the teachings of the present invention, so that the solution proposed here has novelty and inventive activity compared to the state of the art.

Thus, there is a need in the state of the art for new products, through sustainable processes, which are presented as an efficient alternative to enhance hair shine, compared to the main silicones used in cosmetic applications.

### BRIEF SUMMARY OF THE INVENTION

Thus, the present invention solves the problems of the state of the art from a fatty acid derivative, and its use in method of hair treatments, increasing hair shine and strength.

In a first object, the present invention presents a fatty acid derivative, wherein the fatty acid derivative is a hydroxy ester, and the fatty acid comprises from 16 to 22 carbons.

In a second object, the present invention presents a process of producing fatty acid derivatives comprising:
a) a reaction step between a fatty acid with 16 to 22 carbons and an alcohol selected from polyalcohol, wherein the molar ratio of fatty acid:alcohol is from 2:1 to 1:2, and the synthesis takes place for 3 hours to 8 hours at a temperature between 55° C. and 165° C.;
b) a step of water removal;
c) a step of separating the fatty acid derivative.

In a third object, the present invention presents a hair composition comprising 0.1% to 30% by weight of at least one fatty acid derivative, wherein
the fatty acid comprises from 16 to 22 carbons, and
the fatty acid derivative is a hydroxy ester.

In a fourth object, the present invention presents a method of hair treatment comprising a step of applying a fatty acid derivative to the hair, wherein the fatty acid derivative is a hydroxy ester, wherein the fatty acid comprises from 16 to 22 carbons.

These and other objects of the invention will be immediately appreciated by those skilled in the art and will be described in detail below.

### BRIEF DESCRIPTION OF THE DRAWINGS

The following figures are presented:

FIG. 1 shows a graph of shine unit values (Reich-Robbins) of the difference after and before a treatment (1×) of the hair locks of the groups: G1 (Control), G2 (Placebo), G3 (Cyclomethicone), G4 (Phenyltrimethicone) and G5 (Dimethicone), G6 (Amino propyl phenyltrimethicone), G7 (Propylene Glycol Dibenzoate), G8 (ricinoleate mono- and diesters-commercial reference: Proshine). ∘ Significantly higher than Amino propyl phenyltrimethicone groups (p<0.05) and Propylene Glycol Dibenzoate (p<0.05).

FIG. 2 presents the analysis of brightness in bleached hair in shampoo formulations. In figure, * indicates the significant increase compared to control (P<0.001), Placebo (P<0.01) and castor oil (P<0.001). Furthermore, ∘ indicates the significant increase compared to control.

FIG. 3 displays a graph with the results referring to the maximum tension test, with application of the hair composition in the test group after the bleaching process containing ricinoleate mono and diesters (ProShine) (5%). In the figure, G1 represents virgin hair, G2 the bleached hair, G3 the bleached hair treated with a reference product and G4 the bleached hair treated with ricinoleate mono and diesters (ProShine). Finally, the symbol “.” indicates the significant difference in relation to G2, G3 and G4 (P<0.001) groups and the symbol “*” indicates the significant difference in relation to G2 (P<0.05) group.

FIG. 4 displays the graphs with the results referring to the application of the pure product on virgin hair, preferably ricinoleate mono and diesters (ProShine®).

FIG. 5 displays the graphs with the results referring to the application of the pure product on bleached hair, preferably ricinoleate mono and diesters (ProShine®).

FIG. 6 displays the graphs with the results referring to the application of the pure product on dyed hair, preferably ricinoleate mono and diesters (ProShine®).

FIG. 7 shows the photos with the results referring to the application of the pure product on virgin, bleached and dyed hair, preferably ricinoleate mono and diester (ProShine®).

### DETAILED DESCRIPTION OF PREFERRED EMBODIMENTS

Shine actives for hair treatment are mainly made from silicone, due to its refractive, reflection and interaction index with the hair strands. However, silicones are being excluded from natural cosmetic products because they are obtained through chlorinated compounds, that offer environmental danger, as well as issues related to their bioaccumulative properties and because they have low biodegradability.

Furthermore, the synthesis process of the actives of the present invention complies with the precepts of green chemistry, which include the use of catalysts, absence of organic solvents, low energy consumption and atomic economy. For the synthesis of the main chemical structure of the actives, a process was developed that increases the conversion of reagents into products, with optimized chemical proportions, making the process more sustainable.

The present product was developed through a study of the molecular structure of actives considering three elementary properties to obtain a good performance of shine in the hair; refractive index, reflection index and intermolecular interactions between the cuticle of the hair fiber with the chemical functions present in the molecules.

The phenomenon of light refraction can be defined when light passes from one medium to another with a change in propagation speed and is directly related to the reflection index and brightness of a material. Products derived from fatty mono- and diesters (FMDE) have a refractive index (n) similar to phenyltrimethicone, a silicone-based product widely used in the cosmetic area as an active for shine. The refractive index values for the mentioned compounds are n=1.4970 for phenyltrimethicone, n=1.3975 for cyclomethicone and n=1.4790 for FMDE.

In relation to intermolecular interactions, FMDE compounds present polar groups capable of interacting properly on the hair cuticle, thus requiring a lower concentration of these molecules in the formulation, in relation to silicones, to promote the same intensity of shine in the hair.

The synthesis of these molecules for shine involves reactions between fatty acids and/or oils from vegetable sources with diols that react with each other forming fatty mono- and diesters (FMDE). These molecules provide the formation of a film on the hair fiber with a high refractive index, capable of promoting a significant increase in hair shine. One of the benefits most desired by users of cosmetic hair care products.

Given this scenario, a synthetic product with a high degree of vegetalization capable of promoting hair shine in a more accentuated way was developed when compared to the traditional silicones commonly used in the cosmetic industry.

Fatty mono- and diesters (FMDE) are products with a high degree of vegetalization and are synthesized by mild chemical processes. In an embodiment, the product ProShine® under the INCI name Castor Oil Propanediol Esters product containing these compounds, were challenged in a hair treatment against market products, recognized worldwide for being effective in increasing hair shine, that contain silicone derivatives in their composition, and showed equal or greater efficiency, due to a greater interaction of these compounds with the hair fiber.

In addition, it is emphasized that the actives of the present invention are surprisingly able to promote benefits to the user's hair even if used alone, regardless of the presence of silicones and other active components, mainly due to their high interaction with the amino acid residues present in the keratin of the hair fiber, thus maximizing the performance of shine and increased hair strength.

In a first object, the present invention presents a fatty acid derivative, wherein the fatty acid derivative is a hydroxy ester, and the fatty acid comprises from 16 to 22 carbons.

In one embodiment, the fatty acid is a vegetable oil. In one embodiment, the fatty acid is derived from castor oil.

In one embodiment, the fatty acid derivative comprises an alcoholic moiety derived from a polyalcohol. In one embodiment, the polyalcohol is 1,3-propanediol.

In one embodiment, the fatty acid derivative comprises ricinoleate monoester and ricinoleate diester (ProShine®).

In a second object, the present invention presents a process for the production of fatty acid derivatives comprising:
a) a reaction step between a fatty acid with 16 to 22 carbons and an alcohol selected from polyalcohol, wherein the molar ratio of fatty acid:alcohol is from 2:1 to 1:2, and the synthesis takes place for 3 hours to 8 hours at a temperature between 55° C. and 165° C.;
b) a step of water removal;
c) a step of separating the fatty acid derivative.

In one embodiment, the reaction step comprises at least one catalyst selected from the group consisting of ion exchange resin in acid form with an example being Amberlyst 15 (0.5% to 0.6% w/w), methanesulfonic acid (0.5% w/w), lipase catalyst (0.5% w/w) and KOH catalyst (0.5% w/w).

In one embodiment, the reaction of step a) is an esterification.

In one embodiment, the step of separating the fatty acid derivative comprises a step of filtering the catalyst, after the step of removing water, formed by the condensation reaction of step a).

In one embodiment, the fatty acid is derived from castor oil.

In one embodiment, the polyalcohol is a dialcohol. In one embodiment, the dialcohol is 1,3-propanediol.

In a third object, the present invention presents a hair composition comprising 0.1% to 30% by weight of at least one fatty acid derivative, wherein
the fatty acid comprises from 16 to 22 carbons, and
the fatty acid derivative is a hydroxy ester.

In one embodiment, the hair composition comprises from 0.1% to 5% by weight of at least one fatty acid derivative. In one embodiment, the composition comprises from 0.5% to 1% by weight of at least one fatty acid derivative.

In one embodiment, the hair composition comprises ricinoleate monoester and ricinoleate diester (ProShine®).

In one embodiment, the hair composition is a conditioner, leave-in, shampoo, styling cream, hair mask, hair spray, hair care ampoule or finisher.

In a fourth object, the present invention presents a method of hair treatment comprising a step of applying a fatty acid derivative to hair, wherein the fatty acid derivative is a hydroxy ester, wherein the fatty acid comprises from 16 to 22 carbons.

In one embodiment, the present invention presents a method of hair treatment comprising a step of applying to hair a hair composition comprising 0.1% to 30% by weight of at least one fatty acid derivative, wherein
the fatty acid comprises from 16 to 22 carbons, and
the fatty acid derivative is a hydroxy ester.

In one embodiment, the application step is performed for 1 minute to 60 minutes and comprises a subsequent rinsing step. In one embodiment, the application time is from 1 minute to 10 minutes. In one embodiment, the application time is from 1 minute to 5 minutes. In one embodiment, the application time is from 1 minute to 2 minutes.

In one embodiment, the method of treatment is to increase hair shine. In one embodiment, the hair has damage from washing, dyeing, bleaching and heat treatment. In one embodiment, the method of treatment increases the strength of the hairs, or at least maintains the natural strength. In one embodiment, hair strength can be estimated according to the maximum breaking stress of the hair strands.

In one embodiment, the method of hair treatment provides a maximum breaking strength to bleached hair of 1.5·10⁻² Gmf/μm² to 1.8·10⁻² Gmf/μm². In one embodiment, the method of hair treatment provides a maximum breaking strength to bleached hair of 1.6·10⁻² Gmf/μm².

In one embodiment, the method of hair treatment increases the maximum breaking strength of treated hair to values 10% to 15% higher than bleached and untreated hair. In one embodiment, the method of hair treatment increases the maximum breaking strength of treated hair to values 11% to 12% higher than bleached and untreated hair.

In a fifth object, the present invention presents the use of hair compositions comprising said fatty acid derivatives, to increase hair shine and/or maintain hair strength.

In one embodiment, the present invention presents the use of said fatty acid derivatives for the production of hair compositions.

The present invention can be applied to products in the cosmetic and veterinary areas, in particular products for hair care such as conditioners, leave-in, shampoos, hair masks, hair spray, ampoules for hair treatment, finishers, among others, with the objective to clean, protect, modify, style and restore hair.

In the present invention, it is understood by:
Leave-in: as used herein, the terms “Leave-in” and “Leave-on” refer to a product to be applied to the hair, usually after washing the strands, it is a product to be applied to clean hair and without the need to rinse after application.

### EXAMPLES

The examples shown here are intended only to exemplify one of the many ways to carry out the invention, but without limiting its scope.

### Production of Fatty Acid Derivatives

1,3-propanediol was used as dialcohol and fatty acids ranging from C16-C22 were used to synthesize fatty mono- and diesters (FMDE). The reactions were conducted in a temperature range between 55° C. to 165° C.

Non-exhaustive examples of fatty acids that can be used in the present invention are: C16—Palmitic Acid; C18: 1—Oleic Acid; C18: 2—Linoleic Acid; C18: 1 12—OH—Ricinoleic Acid; and C22—Docosanoic Acid.

It is emphasized that the examples of the production process of the assets of the present invention are green, encompassing the use of catalysts, absence of solvent, low energy consumption and atomic economy.

### Example of Production 1

In a 2 L round bottom flask coupled to a mechanical stirrer and under nitrogen flow, 1,3-propanediol (6 mols), fatty acid (4 mols) and ion exchange resin were added as catalyst (0.5% w/w). The reaction system was heated at 100-120° C. for 8 hours under vacuum (35 mbar) to remove the water generated during the process. Subsequently, the reaction system was filtered through a Buchner funnel to remove the catalyst, providing fatty mono- and diesters as products with yields ranging from 70-85%.

### Example of Production 2

In a 2 L round bottom flask coupled to a mechanical stirrer and under nitrogen flow, 1,3-propanediol (6 mols), fatty acid (4 mols) and ion exchange resin were added as catalyst (0.5% w/w). The reaction system was heated at 100-165° C. for 8 hours under vacuum (35 mbar) to remove the water generated during the process. Subsequently, the reaction system was filtered through a Büchner funnel to remove the catalyst, providing fatty mono- and diesters as products with yields ranging from 72-89%.

### Example of Production 3

In a 2 L round bottom flask coupled to a mechanical stirrer and under nitrogen flow, 1,3-propanediol (6 mols), fatty acid (4 mols) and ion exchange resin were added as catalyst (0.6% w/w). The reaction system was heated between 140-165° C. for 3 hours under nitrogen flow. Subsequently, vacuum (35 mbar) was applied for 5 hours. Mono- and fatty diester products were obtained with yields ranging from 75-90%.

### Example of Production 4

In a 2 L round bottom flask coupled to a mechanical stirrer and under nitrogen flow, 1,3-propanediol (6 mols), fatty acid (4 mols) and methanesulfonic acid catalyst (0.5% w/w) were added. The reaction system was heated between 80-90° C. for 3 hours under nitrogen flow and vacuum (150 mbar) for 5 hours. Subsequently, the reaction crude was washed with aqueous sodium carbonate solution and then the product was dried at 90° C. under nitrogen flow and vacuum. The fatty mono- and diester products were obtained in yields between 77-91%.

### Example of Production 5

In a 2 L round-bottomed flask coupled to a mechanical stirrer and under nitrogen flow, 1,3-propanediol (4.5 mols), fatty acid (4 mols) and lipase catalyst, being an example a (CALB) (0.5% w/w). The reaction system was heated between 55-60° C. for 8 hours under nitrogen flow and vacuum (150 mbar). Subsequently, the enzyme was removed by filtration. Fatty mono- and diester products were obtained in yields between 80-93%.

### Example of Production 6

In a 2 L round bottom flask coupled to a mechanical stirrer and under nitrogen flow, 1,3-propanediol (2 mols), vegetable oil, preferably castor oil (4 mols) and KOH catalyst (0.5 mols) were added (0.5% w/w). The reaction system was heated between 55-60° C. for 5 hours under nitrogen flow. Subsequently, the reaction crude was washed with aqueous citric acid solution. Then, residual water was removed from the product by heating at 100° C., under vacuum (100 mbar) and nitrogen flow. The fatty mono- and diester products were obtained in yields between 85-90%.

### Example of Production 7

In a 2 L round bottom flask coupled to a mechanical stirrer and under nitrogen flow, 1,3-propanediol (2 mols), vegetable oil (4 mols), preferably castor oil and lipase catalyst were added (CALB) (0.5% w/w). The reaction system was heated between 55-60° C. for 8 hours under nitrogen flow and vacuum (150 mbar). Subsequently, the enzyme was removed by filtration. The fatty mono- and diester products were obtained in yields between 80-93%.

The examples of production 1 to 3 involving an ion exchange resin in acid form, being the Amberlyst 15 an example of this type of catalyst, have similar yields when compared to homogeneous catalysts (example 4). However, the production process using ion exchange resin does not involve neutralization and washing steps, which leads to a direct and environmentally attractive process.

In addition, synthetic processes involving lipases, preferably CALB, occur under mild reaction conditions, in high yields and with easy removal, because it is a heterogeneous catalyst, showing a great alternative to the classic processes found in the literature.

Examples of cosmetic compositions developed with addition of hydroxy ester of a fatty acid comprising from 16 to 22 carbons are:

Shampoo with Sulfate

Composition comprising: 0.1-30%, preferably 0.1-5%, of at least one fatty acid derivative comprising from 16 to 22 carbons, said derivative being hydroxy ester; sodium lauryl ether sulfate 22-30%, disodium EDTA 0.05-0.1%, cocoamidopropyl betaine 4-8%, amide 90 2.5-5%, sodium chloride 0.5-2.0%, preservatives and antimicrobial agents 0.5-1% and water qsp 100%.

Shampoo without Sulfate

Composition comprising: 0.1-30% preferably 0.1-5% of at least one fatty acid derivative comprising from 16 to 22 carbons, said derivative being hydroxy ester; glycerin 1-3%, sodium lauroyl sarcosinate 1.5-3%, lauryl glucoside 7.5-12%, cocoamidopropyl betaine 4-8%, sodium chloride 0.5-2.0%, preservative and antimicrobial agents 0.5-1% and water qsp 100%.

### Conditioner

Composition comprising: 0.1-30% preferably 0.1-5% of at least one fatty acid derivative comprising from 16 to 22 carbons, said derivative being hydroxy ester; 30/70 cetostearyl alcohol 2.5-5%, cetyl alcohol 1-3%, behenyl trimethyl ammonium chloride 0.3-1.5%, disodium EDTA 0.05-0.1%, cetyl trimethyl ammonium chloride CTAC 0.5-3%, preservative and antimicrobial agents 0.25-1% and water qsp 100%.

### Leave On

Composition comprising: 0.1-30% preferably 0.1-5% of at least one fatty acid derivative comprising from 16 to 22 carbons, said derivative being hydroxy ester; cetostearyl alcohol 30/70 3-5%, cetyl alcohol 0.8-2.5%, disodium EDTA 0.05-0.1%, cetyl trimethyl ammonium chloride CTAC 0.8-2.5%, preservatives and antimicrobial agents 0.25-1% and water qsp 100%.

### Mask

Composition comprising: 0.1-30% preferably 0.1-5% of at least one fatty acid derivative comprising from 16 to 22 carbons, said derivative being hydroxy ester; cetostearyl alcohol 30/70 3-4%, cetyl alcohol 1-2%, behenyltrimethyl ammonium chloride 0.3-1%, disodium EDTA 0.05-0.1%, cetyl trimethyl ammonium chloride CTAC 0.5-2%, preservative and antimicrobial agents 0.25-1% and water qsp 100%.

### Shine Evaluation

The tests for evaluating the effectiveness in the shine of locks of hair were carried out with standardized fibers of CaucasianU, brown, straight hair, supplied by International Hair Importers—USA, model most used for these types of tests (Table 1).

TABLE 1
Description of treatments

| Groups | Treatments |
|---|---|
| G1 | Hair locks washed with sodium lauryl ether sulfate (SLES) - |
| | Control. |
| G2 | Hair locks treated with Placebo Conditioner |
| G3 | Hair locks treated with Conditioner with Cyclomethicone (1%) |
| G4 | Hair locks treated with Conditioner with |
| | Phenyltrimethicone (1%) |
| G5 | Hair locks treated with Conditioner with |
| | Dimethicone (1%) |
| G6 | Hair locks treated with Conditioner with Aminopropyl (1%) |
| G7 | Hair locks treated with Conditioner with Propilene Glycol |
| | dibenzoate (1%) |
| G8 | Hair locks treated with Conditioner with Ricinoleate mono- |
| | and diesters (ProShine) (1%) |

### Pre-Treatment of Locks

The locks were washed with anti-residue shampoo and dried naturally (24±1 h) before treatment.

### Application of Formulations

The formulations were applied according to the protocol described below: For the locks in group G1 (Control), shampoo was applied in a standardized amount of SLES solution (8.1% active) on wet hair and gently massaged for 1 minute. Then rinsed for 1 minute.

For locks in group G2 (Placebo) and with the actives: G3 (Cyclometicone), G4 (Phenyltrimethicone), G5 (Dimethicone), G6 (Aminopropyl phenyltrimethicone) and G7 (Propylene glycol dibenzoate) and G8 (Ricinoleate mono- and diesters—ProShine®), shampoo was applied in a standardized amount of placebo/standard shampoo to wet hair and gently massaged for 1 minute. Then rinsed for 1 minute. After rinsing the hair, the conditioner (3.0±0.2 g) was applied to the entire length of the hair, massaging and untangling the hair. Leaving the product to act for 2 minutes and it was rinsed for 1 minute. This protocol was repeated once, and then four more times, for all groups according to their specific treatment, described in Table 1.

### Efficacy Evaluation on the Shine of Locks of Hair

The locks were kept in a controlled environment at 23±2° C. and 50±5% relative humidity (R.H.), for at least 24 hours before the tests were started. The entire test period was carried out under the same environmental conditions.

The evaluation of hair shine was performed on three locks per group, using the equipment SAMBA Hair System of the company Bossa Nova Technologies, USA.

The locks of hair were positioned on a cylindrical support that slides into the interior of the equipment, in order to carry out the measurements.

The equipment successively captures two parallel images of the locks of hair. Specular image and diffuse image. The specular image is formed only by the light reflected by the hair surfaces (first and second) of reflection. This image contains the brightness information. The diffused image (diffuse) is formed only by the scattered light coming from inside the hair.

The SAMBA system program calculates parameters associated with the reflection and refraction of light, the most used parameter is Luster, which evaluates the real state and quality of the brightness of the object under study. This parameter can be calculated simultaneously through different formulas, and for this study the analysis data obtained by the Reich-Robbins formula was used.

### Statistical Evaluation

For the statistical evaluation of the tests, analysis of variance (ANOVA) was used. Tukey's test was applied when analysis of variance detected significant differences between the groups. For comparative tests between before and after the same sample, the t-Test (Student) was used.

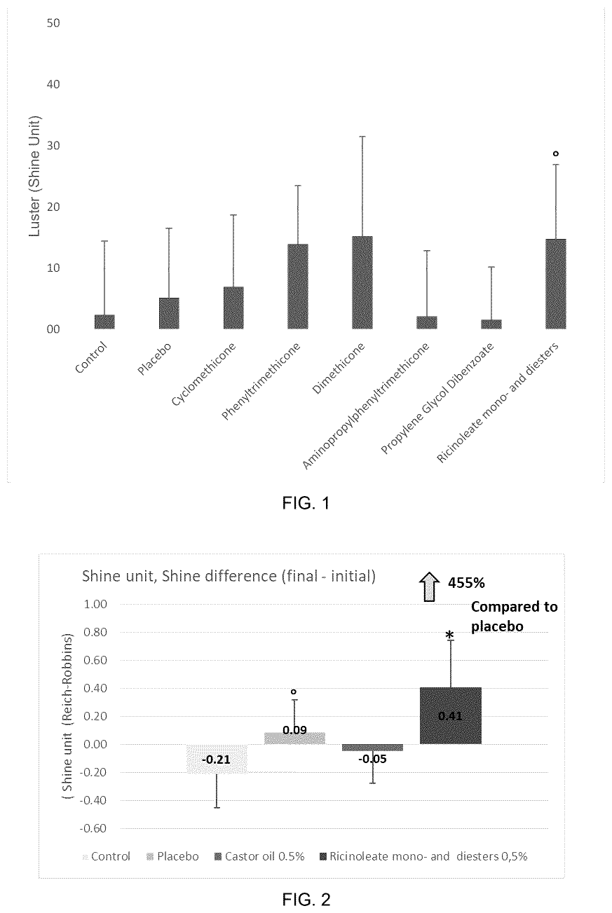

Through FIG. 1 it was possible to observe that the group of locks treated with the ricinoleate mono- and diester actives (ProShine®) obtained a significant increase in the shine value of 85.8% in relation to the group of locks treated with the active Amino phenyltrimethicone and of 89.2% for the group treated with Propylene glycol dibenzoate.

### Comparison of Shine in Shampoo Formulations

Shampoo formulations were applied according to the methodology described above to different hair samples. The difference between the shine provided by the active ricinoleate mono- and diester (ProShine®) (0.5%) compared to castor oil (0.5%), placebo and control can be seen in FIG. 2.

The results allowed evaluating that the active of the present invention presented 455% more shine to the hair when compared with the placebo composition. Furthermore, the active of the present invention provided a greater shine to the hair when compared to the control samples and samples treated with castor oil.

Comparison of Shine with Pure Active

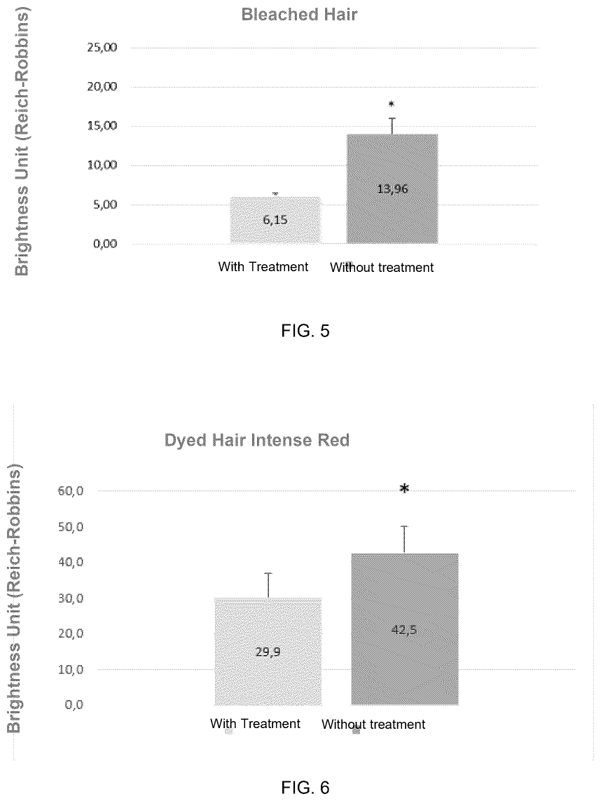

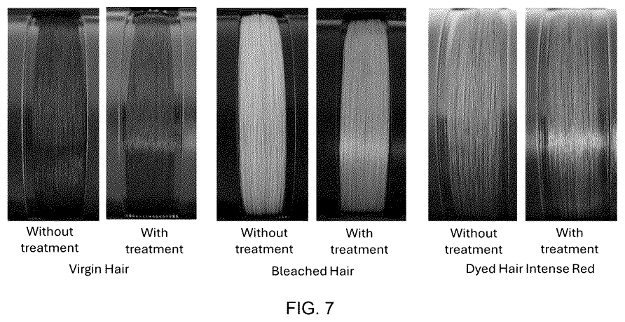

Additionally, tests were carried out with the fatty acid derivatives in their pure form. The application method and shine evaluation were carried out as described above. The locks used were virgin, bleached and dyed (intense red) hair, and the results can be seen in FIGS. 4 to 7.

As can be seen, the treatment of hair with the pure derivative was able to increase the shine of all types of hair tested.

### Evaluation of the Refractive Index

The index of refraction of a substance is the value obtained by the ratio of the speed of light in air and the speed of light in the substance. The equipment used was a refractometer, and the procedure consisted of checking the equipment with purified water, drying the prism carefully with fine paper and placing a drop of the sample on the prism, adjusting the meniscus and recording the result with 4 decimal places after the comma.

TABLE 2
Refractive indexes.

| Compound | Refractive Index (n) |
|---|---|
| FMDE | 1.4790 |
| Phenyltrimethicone | 1.4970 |
| Cyclomethicone | 1.3975 |

### Resistance Test Comparing Treated and Virgin Hair

### Bleaching Process

A bleaching mixture was prepared with 30 g of bleaching powder and 60 g of hydrogen peroxide 30 volumes, said mixture was uniformly applied to locks of hair (with an average weight of 2.0 g per lock).

After application, the locks were wrapped in aluminum foil and kept for 30 minutes in the Suntest chamber at 40° C. After the time for the bleaching mixture to work, the locks were washed and rinsed thoroughly with running water for 2 minutes and then dried using a dryer at medium temperature. The process in question was repeated two more times, totaling three bleachings.

### Application of Hair Composition

After the bleaching process, leave-in compositions, shampoo and conditioner were applied to the locks.

A standardized amount of Leave-in (with 5% ricinoleate mono- and diesters-ProShine) was applied to wet hair. The locks of hair were gently massaged and left to rest for 30 minutes with the product in the hair, then rinsed for 1 minute.

Then, after being rinsed, a standardized amount of anti-residue shampoo (SLES solution with 8.1% active) was applied to wet hair. After applying the shampoo, the locks of hair were gently massaged for 1 minute and rinsed for 1 minute.

Finally, after rinsing, conditioner was applied over the entire length of the hair, massaging and untangling the hair for 2 minutes. In a last step, the locks were rinsed for 1 minute.

This treatment was repeated two more times, totaling three treatments. However, in the last treatment, after the application of the Leave-in (with 5% of ricinoleate mono- and diester—ProShine®), the excess of the product was removed, without a subsequent application of shampoo and conditioner. The removal of the product was necessary to carry out the resistance test, since the presence of excess makes it difficult to carry out the tests by making the crimps slippery.

### Resistance Test Method

The hair fibers were kept in a room with a controlled temperature of 22±2° C. and relative humidity of 25±5%, for at least 24 hours, and the same conditions were maintained during the test.

For the evaluation of mechanical properties, samples of hair fibers were randomly selected from each group. The samples referring to each treatment were submitted to the reading of the diameter of each strand with the equipment FDAS-770 Dia-Stron Ltd and subsequently to the tensile test using the equipment MTT-680—Automated Miniature Tensile Tester da Dia-Stron Ltd. Through the software of the equipment, the Maximum Tension parameter resisted by the fiber before rupture was evaluated, correlating the maximum force with the diameter of the hair fiber.

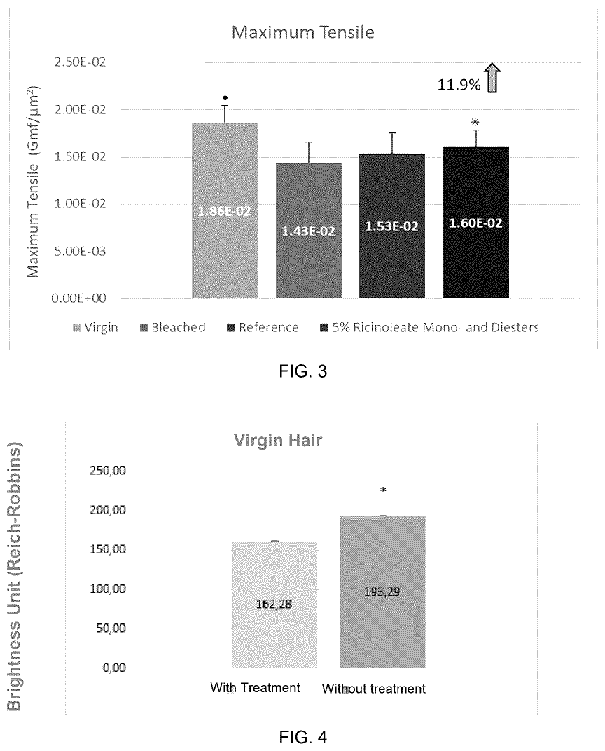

The results referring to the resistance test can be seen in FIG. 3. In short, it was verified that the hair fibers that underwent a bleaching process and were treated with Leave-in plus 5% of ricinoleate mono- and diesters-ProShine® (G4), presented significant increase of 11.9% compared to fibers that were bleached and not treated with ricinoleate mono- and diesters-ProShine® (G2), at the maximum resisted tensile values before failure. The increase in the maximum tensile values indicates the increase in the resistance of the hair fibers.

The increase in hair fiber resistance can be explained due to the intermolecular interactions that occur between the ricinoleate mono- and diester active (ProShine®) with the residues of the side chains of amino acids present in keratin. These interactions promote the formation of cross-links, which promote increased strength in the hair, indicating that there is permeation of the active in the hair fiber. Thus, the ricinoleate mono- and diester active (ProShine®), in addition to promoting intense shine, promotes increased strength in the hair.

Those skilled in the art will value the knowledge presented here and will be able to reproduce the invention in the presented embodiments and in other variants and alternatives, covered by the scope of the following claims.

What is claimed is:

1. A fatty acid derivative comprising from 16 to 22 carbons, wherein the fatty acid is a hydroxy ester.

2. The fatty acid derivative according to claim 1, wherein the derivative is ricinoleate mono- and diesters.

3. A production process of a fatty acid derivative, as defined according to claim 1, comprising the steps of:
a) a reaction step between a fatty acid having 16 to 22 carbons and an alcohol selected from the group consisting of polyalcohol, in which the molar ratio of fatty acid:alcohol is from 2:1 to 1:2, and the synthesis occurs for 3 hours to 8 hours at a temperature between 55° C. to 165° C.;
b) a step of water removal; and
c) a step of separating the fatty acid derivative.

4. The production process according to claim 3, wherein the reaction step (a) comprises at least one catalyst selected from the group consisting of an ion exchange resin in acid form (0.5% to 0.6% w/w), methanesulfonic acid (0.5% w/w), lipase catalyst (0.5% w/w), and KOH catalyst (0.5% w/w).

5. The production process according to claim 3, wherein the polyalcohol is 1,3-propanediol.

6. A hair composition comprising from 0.1% to 30% by weight of at least one fatty acid derivative according to claim 1.

7. The hair composition according to claim 6, wherein the hair composition comprises from 0.1% to 5% by weight of at least one fatty acid derivative.

8. The hair composition according to claim 6, wherein the hair composition is a conditioner, leave-in, shampoo, hair mask, styling cream, hair spray, ampoule for hair treatment, or finisher.

9. A method of hair treatment, comprising applying a fatty acid derivative, as defined according to claim 1, to the hair.

10. The method of hair treatment, according to claim 9, wherein the fatty acid derivative is comprised in a hair composition comprising from 0.1% to 30% by weight of at least one fatty acid derivative comprising from 16 to 22 carbons, wherein the fatty acid is a hydroxy ester.

11. The method of hair treatment, according to claim 9, wherein the application step is carried out for 1 minute to 5 minutes and comprises a subsequent rinsing step.

12. The method of hair treatment according to claim 9, wherein the method is for increasing the shine of the hair and/or increasing the strength of the hair.
